(19) United States
(12) Patent Application Publication
(10) Pub. No.: US 2022/0336059 A1
(43) Pub. Date: Oct. 20, 2022

(54) SYSTEMS AND METHODS FOR IDENTIFYING RECIPES FOR BATCH TESTING

(71) Applicant: Automat Solutions, Inc., Fremont, CA (US)

(72) Inventors: Xiaoliang Wang, Alameda, CA (US); Naixin Zong, Katy, TX (US); Xuejun Wang, Pleasanton, CA (US)

(21) Appl. No.: 17/711,024

(22) Filed: Mar. 31, 2022

Related U.S. Application Data
(60) Provisional application No. 63/174,532, filed on Apr. 13, 2021.

(51) Int. Cl. G16C 60/00; G16C 20/80; G16C 20/10; G16C 20/70

(52) U.S. Cl. CPC G16C 60/00; G16C 20/80; G16C 20/10; G16C 20/70

(57) ABSTRACT

Disclosed are systems and methods for generating candidate recipes for batch testing battery recipes in robotics laboratory equipment. In one embodiment, the candidate recipes in a batch, share the maximum number of chemicals in common, while as a batch, they utilize a minimum number of chemicals. The candidate recipes are identified by constructing a graph where an initial selection of recipes are placed at each node. The graph yields the candidate recipes in the batch.

### CROSS-REFERENCE TO RELATED APPLICATIONS

This application claims the benefit of U.S. Application No. 63/174,532 filed on Apr. 13, 2021, which is incorporated herein by reference in its entirety.

### STATEMENT REGARDING FEDERALLY SPONSORED R&D

The inventions disclosed herein were made with government support under Grant No. 1938253 awarded by the National Science Foundation (NSF). The government has certain rights in the invention.

### FIELD

This application relates generally to the field of manufacturing optimization and in particular to finding groupings of materials to make conducting experimentation and discovering advanced materials more efficient.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

In various material science fields, experimenting with various recipes may be needed to determine whether one or more combinations of chemicals result in discovery of more advanced material with certain desirable properties.

### SUMMARY

The appended claims may serve as a summary of this application.

### BRIEF DESCRIPTION OF THE DRAWINGS

These drawings and the associated description herein are provided to illustrate specific embodiments of the invention and are not intended to be limiting.

FIG. 1 illustrates a diagram of an artificial intelligence model according to an embodiment.

FIG. 2 illustrates a diagram of identifying and outputting candidate recipes in a batch for laboratory, robotics and/or human analysis.

FIG. 3 illustrates a flowchart of a method of outputting candidate recipes for batch testing.

FIG. 4 illustrates an environment in which some embodiments may operate.

### DETAILED DESCRIPTION

The following detailed description of certain embodiments presents various descriptions of specific embodiments of the invention. However, the invention can be embodied in a multitude of different ways as defined and covered by the claims. In this description, reference is made to the drawings where like reference numerals may indicate identical or functionally similar elements.

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail. When the terms “one”, “a” or “an” are used in the disclosure, they mean “at least one” or “one or more”, unless otherwise indicated.

Artificial intelligence (AI) has experienced a tremendous growth and has contributed immensely to various technological fields. In many cases, AI models, such as machine learning models, receive a training dataset, finetune their internal weights and parameters and subsequently make predictions on unknown datasets. In some technical fields, the AI predictions are tested in laboratory environments and the results are used as new training dataset to further finetune the AI models and better their predictions. Example fields, where such an iterative process is deployed, include small molecule identification and science, material science, robotics, drug development and other fields where AI is applied. Often the output of the AI models, at least in the initial stages, can include predictions in the form of voluminous datasets. It may not be practical to take a voluminous AI output and test it in laboratory setting. Instead, an intelligent subset of the output of the AI models should be chosen and experimented with, in order to further finetune the AI models.

In one example, AI can be applied to finding optimized battery and secondary storage recipes, where the AI assists in arriving at combination of chemicals or recipes which can yield various desirable properties in battery technology, such as superior conductivity, longevity, or other suitable properties. The designer of a battery may use computer or AI models to generate battery recipes from a pool of chemicals. While the pool of chemicals can be a finite set, the combination of those chemicals and potential recipes can be several hundred orders of magnitude larger.

Consequently, the total number of possible recipes can be impractical to test in laboratory settings. In some AI fields, various techniques other than laboratory experimentation may be applicable to further finetune the AI models. These methods can include, manual auditing or human examination of results, secondary computer models, statistical analysis, comparison with empirical data and others. Regardless of the method used to examine the output of the AI models, these methods can be inadequate or impractical in the case of large outputs from the AI models. As a result, there is a need for systems and methods that intelligently reduce the size of an AI model output. The embodiments described herein can address this need. It is noted that although the examples used in this document include recipes generated from AI models, the described embodiments are also applicable to batch selection of recipes generated by other methods, including but not limited to, brute force sampling, statistical sampling methods (probability sampling, stratified sampling, etc.), snowball sampling and others. For illustration and understanding, the embodiments are described in the context of battery manufacturing and choosing the recipes and chemicals for that purpose; however, the described technology is not limited to those applications. Persons of ordinary skill in the art can readily extend the principles described herein to other technological fields.

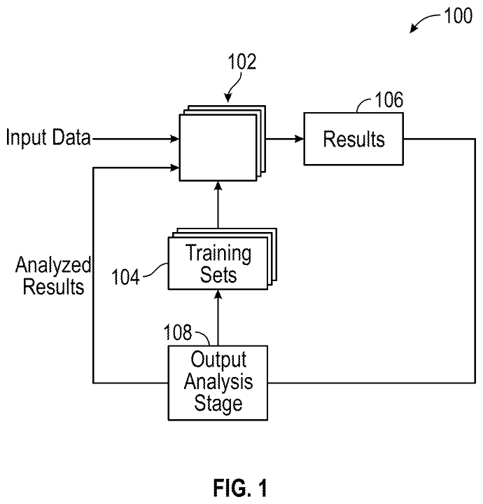

FIG. 1 illustrates a diagram 100 of an artificial intelligence model 102. The AI model 102 can include any number of AI operations, such as machine learning, deep learning, neural networks, convolutional neural networks (CNNs) or others. The AI model 102 can be trained using training sets 104. The model 102 can then receive unknown input data and perform predictions, identifications, or other AI operations on the unknown input data, outputting results 106. The results 106 can be analyzed by an output analysis stage 108, where the accuracy, applicability, correctness, or other selected characteristics that are desired to be seen in the output of the AI model 102 are analyzed. The analyzed results can then be iteratively used in the AI model 102 as improved training sets or as input data to further optimize the AI model 102.

As described earlier, the results 106 can be a large dataset, which may need to be further reduced in size in order to be efficiently processed in the output analysis stage 108. As an example, the output analysis stage 108 can be hardware and/or software which can test battery recipes output by the AI model 102 in batches and by using robotics and/or human operators. And this may be the case, with other approaches to AI output analysis techniques, where hardware/software and/or human operator can batch test the output of the AI one batch at a time and use the result to finetune the AI models.

In the context of battery manufacturing and discovering efficient materials for various battery components, an approach can include testing a collection of battery recipes in a laboratory setting, using robotics or human operators. Whether robotics or human operators are used, the testing system can process a batch or a collection of recipes at a time. The time it takes to prepare each batch for testing can be considerable or can otherwise reduce the efficiency of discovering desirable battery recipes and combination of chemicals. The testing preparation time can be reduced if the recipes in a collection can share a maximum number of chemicals between them, while the total number of chemicals used in the collection of recipes overall is minimized. The described embodiments enable systems and methods that can select a batch or collection of recipes for laboratory testing in a manner that the testing preparation time is minimized. For example, the described systems and methods can generate and/or receive recipes and output a collection or batch of recipes for testing, in a manner that the recipes in the collection share a maximum number of chemicals in common, while the total number of chemicals used in the collection of the recipes is minimized.

At the same time, the available starting chemicals for a battery recipe may be drawn from a pool of chemicals S. For example, the pool of chemicals S can include chemicals C1-C300. Persons of ordinary skill in the art can envision a pool of chemicals of different sizes depending on the application. In one embodiment, random recipes can be generated from the pool of chemicals S by choosing various combinations of chemicals from the pool of chemicals S. In other embodiments, recipes can be generated via AI, for example using the system described in FIG. 1. Additionally, recipes can be generated with some preliminary constraints. For example, the recipe size (e.g., the number of chemicals in each recipe) can be selected to be a constant parameter M. Another constraint can include forcing an essential chemical set (ECS) into each randomly generated recipe. This constraint can be useful if there are some essential combination of chemicals from the pool of chemicals S that are selected to be included in each recipe. For example, in some battery recipes, there are some known and common chemicals that are required in each battery recipe. Accordingly, in some cases, the recipes are randomly generated or otherwise forced to include at least a set of essential chemicals (ECS). Given these initial constraints, a preliminary set of recipes (PSR) can be generated.

In some cases, the PSR can be filtered based on some filtering criteria to reduce the size of the PSR for further processing. In one example, a frequency table can be used to filter out recipes that include infrequently occurring or rare chemicals. To build the frequency table, a table having S cells, corresponding to each chemical in the pool of chemicals S can be generated. Each cell can hold a counter corresponding to the cell's chemical. Then, each recipe is parsed and for each chemical encountered, the corresponding counter is incremented accordingly. The resulting table yields the frequency of occurrence of each chemical in the recipes at hand. Subsequently, chemicals occurring below a selected threshold can be designated rare or infrequently occurring. The frequency table can be used to filter the PSR. In other embodiments, the PSR can be filtered based on a selected table of rare chemicals, defined by some other criteria, for example, based on outside knowledge. For example, in the context of battery recipe generation, a chemist can define a list of rare or infrequently occurring chemicals based on which the PSR can be filtered.

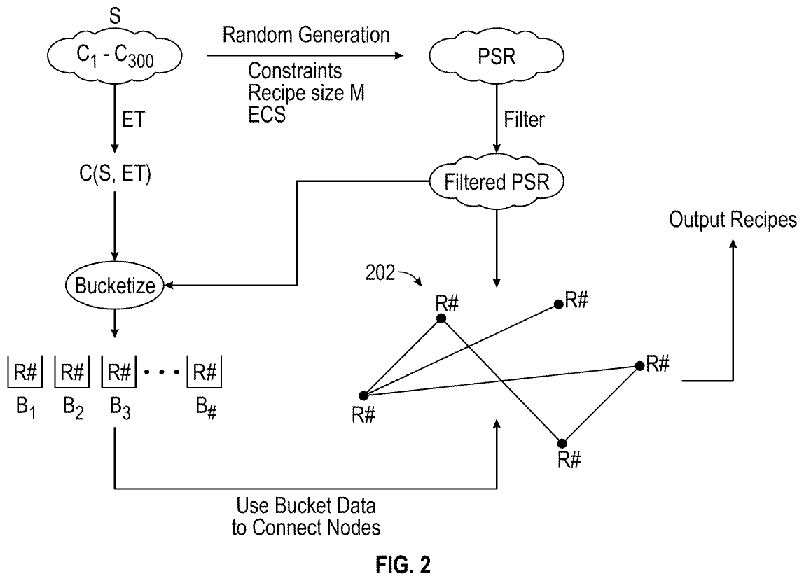

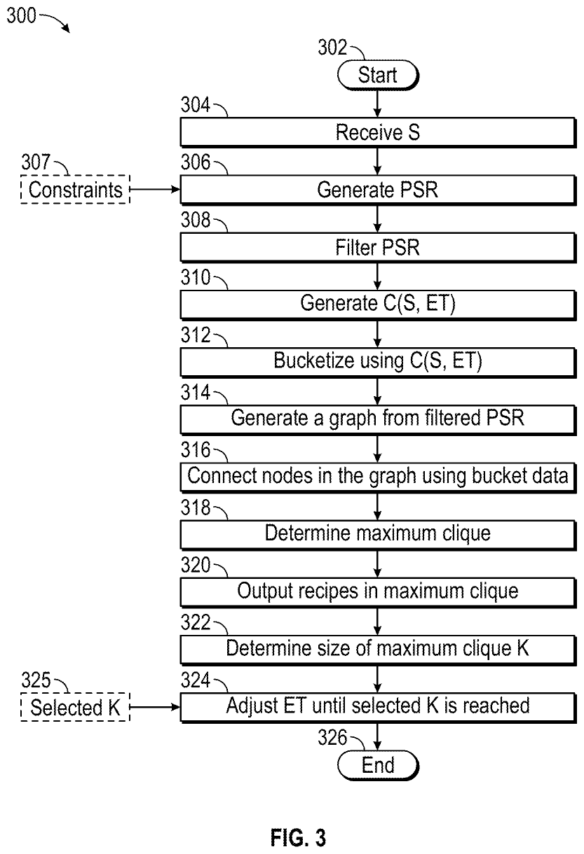

The pool of chemicals S can also be used to bucketize the recipes based on an edge threshold (ET) parameter. The term edge threshold ET is further described in relation to the embodiments of FIGS. 2 and 3. Buckets are generated, where each bucket corresponds to a combination of ET chemicals from the pool of chemicals S. To bucketize the recipes, the recipes are parsed and tagged with a corresponding bucket number if the bucket's combination is found in the recipe. Accordingly, each bucket identifies recipes that share the same ET chemicals in common.

Next, a graph (V,E) is generated where each node of the graph is a recipe from the filtered PSRs. In some embodiments, the filtering step can be skipped, and each node of the graph includes a recipe from the PSR. The bucket data can then be used to connect an edge between the nodes that share at least the ET number of chemicals in common. The graph can be generated with average case complexity of

$$O ( C ( S , ET ) \cdot C ( ECS , ET ) \cdot N \cdot ET + \frac{{N}^{2} log ( N ) * {C ( ECS , ET )}^{2}}{C ( S , ET )} )$$

Where N is the number of recipes in a batch or collection, S is the number of chemicals in the pool of chemicals S, C(a,b) is a combination of a choose b, ECS is the number of chemicals in the essential chemicals set and ET is the edge threshold, or the minimum number of chemicals shared between two recipes. Case complexity in this scenario refers to the levels of complexity a computer system needs to resolve in order to generate the graph, as described herein.

The graph can be used in a variety of ways to obtain candidate recipes for experimentation or analysis, where the collection of the recipes shares a maximum number of chemicals in common, while the total number of chemicals used between the candidate recipes is minimized.

In one embodiment, a maximum clique of the graph can be obtained. The recipes in the maximum clique have the following property: for every pair of vertices in the maximum clique, the intersection will be greater or equal to a given threshold size of the maximum clique K. Therefore, the total union of N nodes of M chemicals each with the threshold of K, will be less than or equal to M+(N−1)·(M−K)

The maximum clique of the graph can be obtained by a variety of methods. In one embodiment, for example, the maximum clique can be obtained by applying MaxCliqueDyn developed by Janez Knoc, optimized using an internal approximator color sort, resulting in reduced average case computational complexity, in comparison with the standard Bron-Kerbosch algorithm maximum clique of size K algorithm with runtime complexity of 0(3n/3). In cases, where the graph is sparse (e.g., the number of edges is relatively low compared to a fully connected graph, where each node is connected), the described approach can handle 102,000 or more recipes in manageable time.

In some embodiments, the edge threshold can be adjusted until the maximum clique yields a preselected size K, having K recipes in the maximum clique. This can be helpful if the number of recipes in a batch is constrained by some external factor, such as the capacity of a robotic testing module, or constraints on preparation, testing and analysis time of K recipes at a time.

Table 1 below, illustrates runtime experiments using the systems and methods described above, on randomly generated recipes with each recipe having at least 6 ECS, as well as other chemicals from a pool of chemicals S of size 300.

TABLE 1

| | Initial Set | Filtered | Remain | Similarity | Max | | Runtime (i9 |
|---|---|---|---|---|---|---|---|
| | amount | amount | amount | threshold | Clique | Union | 9980 k, OFast) |
| Experiment 1 | 1000 | 800 | 200 | 1 | 12 | 56 | 0.497 milis |
| Experiment 2 | 1000 | 600 | 400 | 1 | 18 | 82 | 2.675 milis |
| Experiment 3 | 1000 | 400 | 600 | 1 | 27 | 107 | 5.463 milis |
| Experiment 4 | 1000 | 0 | 1000 | 1 | 33 | 125 | 17.004 milis |
| Experiment 5 | 1000 | 800 | 200 | 2 | 3 | 14 | 0.118 milis |
| Experiment 6 | 1000 | 600 | 400 | 2 | 4 | 18 | 0.488 milis |
| Experiment 7 | 1000 | 400 | 600 | 2 | 5 | 22 | 0.764 milis |
| Experiment 8 | 1000 | 0 | 1000 | 2 | 5 | 22 | 2.148 milis |
| Experiment 9 | 1000 | 0 | 1000 | 3 | 2 | 9 | 1.714 milis |
| Experiment 10 | 4000 | 3800 | 200 | 1 | 13 | 60 | 0.804 milis |
| Experiment 11 | 4000 | 3600 | 400 | 1 | 21 | 78 | 3.352 milis |
| Experiment 12 | 4000 | 3200 | 800 | 1 | 36 | 126 | 14.759 milis |
| Experiment 13 | 4000 | 2400 | 1600 | 1 | 55 | 166 | 72.114 milis |
| Experiment 14 | 4000 | 800 | 3200 | 1 | 91 | 226 | 413.518 milis |
| Experiment 15 | 4000 | 3800 | 200 | 2 | 4 | 16 | 0.127 milis |
| Experiment 16 | 4000 | 3200 | 800 | 2 | 5 | 22 | 1.488 milis |
| Experiment 17 | 4000 | 2400 | 1600 | 2 | 6 | 24 | 5.067 milis |
| Experiment 18 | 4000 | 800 | 3200 | 2 | 8 | 32 | 16.72 milis |
| Experiment 19 | 4000 | 3800 | 200 | 3 | 2 | 9 | 0.088 milis |
| Experiment 20 | 4000 | 3200 | 800 | 3 | 2 | 9 | 1.114 milis |
| Experiment 21 | 4000 | 2400 | 1600 | 3 | 2 | 9 | 3.895 milis |
| Experiment 22 | 4000 | 800 | 3200 | 3 | 3 | 12 | 12.674 milis |
| Experiment 23 | 16,000 | 15,800 | 200 | 1 | 16 | 62 | 0.934 milis |
| Experiment 24 | 16,000 | 15,600 | 400 | 1 | 27 | 95 | 3.952 milis |
| Experiment 25 | 16,000 | 14,400 | 1600 | 1 | 68 | 161 | 87.354 milis |
| Experiment 26 | 16,000 | 9600 | 6400 | 1 | 185 | 246 | 3295.04 milis |
| Experiment 27 | 16,000 | 15,800 | 200 | 2 | 5 | 22 | 0.154 milis |
| Experiment 28 | 16,000 | 15,600 | 400 | 2 | 5 | 22 | 0.475 milis |
| Experiment 29 | 16,000 | 14,400 | 1600 | 2 | 8 | 32 | 4.496 milis |
| Experiment 30 | 16,000 | 9600 | 6400 | 2 | 13 | 49 | 66.11 milis |
| Experiment 31 | 16,000 | 15,800 | 200 | 3 | 2 | 9 | 0.087 milis |
| Experiment 32 | 16,000 | 15,600 | 400 | 3 | 3 | 12 | 0.318 milis |
| Experiment 33 | 16,000 | 14,400 | 1600 | 3 | 3 | 12 | 3.197 milis |
| Experiment 34 | 16,000 | 9600 | 6400 | 3 | 3 | 11 | 51.946 milis |
| Experiment 35 | 160,000 | 159,600 | 400 | 1 | 33 | 85 | 5.255 milis |
| Experiment 36 | 160,000 | 158,400 | 1600 | 1 | 89 | 132 | 171.435 milis |
| Experiment 37 | 160,000 | 153,600 | 6400 | 1 | 253 | 173 | 6288.5 milis(6s) |
| Experiment 38 | 160,000 | 159,600 | 400 | 2 | 6 | 25 | 0.64 milis |
| Experiment 39 | 160,000 | 158,400 | 1600 | 2 | 10 | 38 | 6.277 milis |
| Experiment 40 | 160,000 | 153,600 | 6400 | 2 | 18 | 60 | 97.881 milis |
| Experiment 41 | 160,000 | 147,200 | 12,800 | 2 | 26 | 86 | 468.924 milis |
| Experiment 42 | 160,000 | 159,600 | 400 | 3 | 3 | 12 | 0.301 milis |
| Experiment 43 | 160,000 | 158,400 | 1600 | 3 | 3 | 12 | 3.849 milis |
| Experiment 44 | 160,000 | 153,600 | 6400 | 3 | 4 | 15 | 50.484 milis |
| Experiment 45 | 160,000 | 147,200 | 12,800 | 3 | 5 | 18 | 203.573 milis |
| Experiment 46 | 160,000 | 134,400 | 25,600 | 3 | 7 | 24 | 909.99 milis |
| Experiment 47 | 160,000 | 158,400 | 1600 | 4 | 2 | 8 | 3.314 milis |
| Experiment 48 | 160,000 | 153,600 | 6400 | 4 | 2 | 8 | 50.262 milis |
| Experiment 49 | 160,000 | 147,200 | 12,800 | 4 | 2 | 8 | 198.932 milis |
| Experiment 50 | 160,000 | 134,400 | 25,600 | 4 | 3 | 10 | 913.022 milis |
| Experiment 51 | 1,600,000 | 1,593,600 | 6400 | 2 | 30 | 81 | 199.279 milis |
| Experiment 52 | 1,600,000 | 1,587,200 | 12,800 | 2 | 41 | 93 | 1005.53 milis(1s) |
| Experiment 53 | 1,600,000 | 1,574,400 | 25,600 | 3 | 8 | 25 | 896.873 milis |
| Experiment 54 | 1,600,000 | 1,548,800 | 51,200 | 3 | 9 | 29 | 3777.19 milis(3s) |
| Experiment 55 | 1,600,000 | 1,497,600 | 102,400 | 3 | 11 | 34 | 13692.2 milis(13s) |
| Experiment 55 | 1,600,000 | 1,497,600 | 102,400 | 4 | 4 | 12 | 13162.7 milis(13s) |
| Experiment 56 | 1,600,000 | 1,395,200 | 204,800 | 3 | 13 | 37 | 88073.4 milis(88s) |
| Experiment 57 | 1,600,000 | 1,395,200 | 204,800 | 4 | 4 | 11 | 77785.1 milis(77s) |
| Experiment 58 | 1,600,000 | 1,280,000 | 320,000 | 4 | 4 | 12 | 152290 milis(152s) |

In Table 1, the initial set amount refers to the PSR. The filtered amount refers to the filtered PSR. Similarity threshold refers to the edge threshold ET or the minimum number of chemicals shared between the recipes on connected nodes of the graph. The column, Max Clique, refers to the size of the maximum clique or the number of recipes in the maximum clique. The column, Union, refers to the union or the total number of chemicals used in the collection of the recipes in the maximum clique. The collection of recipes in the maximum clique can be considered a batch of recipes taken to the laboratory and/or otherwise analyzed by human or machine operator. For example, the batch can be prepared in the lab and placed in a robotic module for chemical analysis and testing of various selected characteristics and/or parameters of batteries. The column, Runtime (i9 9980k, OFast), refers to the runtime measurement on an Intel® i9 CPU optimized using GCC OFast option.

The computational time varies with the number of recipes and ET. As an example, the trial with 102 thousand recipes and ET=4, found a clique of size 4 in approximately 13 seconds (Experiment 55). Too high of a maximum clique size (K) may be undesirable, as the size of recipes able to be batch tested at a time may be constrained by a laboratory setting, e.g., the size of a robotic testing module, and other external factors.

Experiments can also be run with max-flow-based approach, Heavies-K-Subgraph-based approach, dynamic-programming-based approach and special-clustering-based approach. The Max-flow-based approach (abbreviated), in some cases, is unable to produce the desired K (size of the recipes in a batch) when facing evenly distributed appearance of each chemical in recipes. Hk-based approach is optimized for a reward function squared the amount of appearance a certain chemical in K recipes, which could give inaccurate results in dense graphs when subgraph size is large. Also, Hk-based approach has many times higher time complexity than the approach described herein. Dynamic-Programming-based approaches generally give less accurate results when compared to the approaches described, due to their nature of overlapping sub-case in dynamic-programming-based approaches. Spatial-clustering-based approach gives slightly better results than dynamic programming, but still worse than the described approaches on average. Also, the special-clustering approaches suffer from uncontrollable cluster size unless specially optimized.

Table 2 illustrates a comparison between the densest K subgraph approach and the maximum clique approach as described herein.

TABLE 2

| Execution time | | |
|---|---|---|
| of size/method | Densest K Subgraph | Maximal Clique |
| 5 nodes | 3.1 s, union = 21 | 0.005 s, union = 22 |
| 6 nodes | 65.7 s, union = 25 | 0.007 s, union = 25 |
| 7 nodes | 3879.3 s, union = 29 | 0.007 s, union = 28 |
| 8 nodes | 2230 s, union = 31 | 0.009 s, union = 31 |

In Table 2, the nodes indicate recipes on each node of the graph. The densest K subgraph yields substantially longer runtimes than the maximum clique approach as described herein.

FIG. 2 illustrates a diagram of identifying and outputting candidate recipes in a batch for laboratory, robotics and/or human analysis. The recipes in the batch share a maximum number of chemicals in common, while using the minimum number of chemicals in their union. A pool of chemicals S can include chemicals C1-C300. Other numbers of chemicals are also possible depending on the implementation and/or the application where the described embodiments are deployed. In one embodiment, a PSR is generated using one or more constraints. The constraints for generating the PSR can include recipe size (e.g., M) and an essential chemical set (ECS), which are to be included in every randomly generated recipe. As an example, the ECS of 6 chemicals can include C1-C6 and the random recipe generation instruction for a recipe Rn can be defined as follows:

Rn=choose M number of chemicals from S, while M includes C1-6.

The PSR can be further reduced using one or more filtering techniques, as described above. The filtered PSR can be used to construct a graph 202, where each recipe Rn in the PSR is placed at a node of the graph 202. To further construct the graph, nodes (recipes) sharing a minimum number (ET) of chemicals in common are to be connected. To more efficiently achieve this, the recipes are bucketized and/or tagged with a corresponding bucket number, where each bucket contains recipes sharing at least ET number of chemicals in common. An edge threshold parameter ET is selected and combinations of chemicals of size ET are generated. This can be denoted as C(S,ET). In factorial terms, C(S,ET)=S!/(ET!*(S-ET)!). Each bucket or bucket number B# can correspond to each combination from C(S,ET). Next, the PSR, or the filtered PSR is bucketized or tagged with a bucket number corresponding to a combination from C(S,ET) if the combination is found in the recipe. In one embodiment, the recipes (e.g., the PSR or the filtered PSR) are parsed for each combination and tagged with the combination's bucket number B# if the combination is found in the recipe.

Next, the bucket data can be used to connect the nodes of the graph 202, which share at least ET number of chemicals in common. In one embodiment, the recipes in each bucket are pairwise connected in the graph 202 via an edge. In this manner, each connected node shares at least ET number of chemicals in common. As an example, if bucket 1 includes recipes R1, R2, R6, R10, R23, wherein each recipe shares at least C1, C2 and C3 in common (ET=3), then in graph 202 the nodes corresponding to these recipes can be connected pairwise via an edge.

The recipes at the nodes in the maximum clique of the graph 202 can be candidate recipes, which share a maximum number of chemicals in common, while the union of the overall chemicals used between those recipes is minimized, relative to other candidate recipes not in the maximum clique. Furthermore, the size of the maximum clique (the number of nodes or recipes in the maximum clique) can be a parameter for which adjustment or optimization may be performed. In some cases, the number of recipes that can be batch tested at a time may be constrained by some external factors, such as the size of the robotics testing automation machines. As a result, it may be desirable to achieve at a preselected size of the maximum clique. The size of maximum clique, which for example can be denoted by parameter K, can be related to the edge threshold parameter ET. In this scenario, ET can be adjusted until the preselected size of the maximum clique K can be achieved. For example, referencing Table 1, maximum clique sizes of less than 20 are more practical and desirable for laboratory testing, and as a result a corresponding ET is chosen to achieve the selected size of the maximum clique.

FIG. 3 illustrates a flowchart of a method 300 of outputting candidate recipes for batch testing, where the chemicals shared between the recipes are maximized, while the union of chemicals used between the recipes is minimized. The method 300 starts at step 302. At step 304, a pool of chemicals S is received. At step 306, a preliminary set of recipes PSR is generated by generating combinations of chemicals from S. In some embodiments, the recipe generation step 306 is performed by taking constraints 307 into account. Example constraints 307 can include, each recipe be of size M (e.g., have M number of chemicals in each recipe), and each recipe includes an essential chemical set ECS. In some embodiments, the PSR is received from an external source (e.g., from an AI module results 106). In these scenarios, there is no random generation of recipes to generate PSR. In some embodiments, the external recipes can be culled based on the constraints 307 to generate the PSR. Still in other embodiments, the PSR can be directly received from an external source, where no random generation and no enforcing of constraints 307 are performed. Next, at step 308, the PSR is filtered as described above, for example, to exclude recipes that use rare chemicals. In some embodiments, the filtering step 308 may be skipped and can be optional. In these cases, the remaining steps of method 300 can be performed on the PSR.

Using a selected parameter edge threshold ET, at step 310, combinations of ET chemicals from S (e.g., C(S,ET)) are generated. At step 312, the combinations C(S,ET) is used to generate a plurality of buckets of recipes, wherein the recipes in each bucket share at least ET number of chemicals in common. As an example, each recipe Rn can be parsed relative to each combination or bucket B# and if B# is found in Rn, Rn can be tagged as belonging to bucket B#. In this manner, the recipes are bucketized, where the recipes in each bucket share at least in common the combination of chemicals corresponding to that bucket. Other methods of tagging or bucketizing the recipes relative to the combinations C(S,ET) can also be used.

At step 314, a graph is generated from the PSR or the filtered PSR, where each node of the graph includes a recipe. At step 316, the bucket data is used to connect the nodes of the graph. Referencing each bucket, the recipes in the bucket are pairwise connected. In other words, the nodes having at least ET chemicals in common are connected in the graph. At step 316, a maximum clique of the graph is determined. At step 320 the recipes in the nodes of the maximum clique are outputted. As described earlier, the recipes in the maximum clique share the maximum number of chemicals in common, while their union uses the minimum number of chemicals.

The size of the maximum clique can indicate the number of recipes that are to be batch tested together. In some embodiments, it is advantageous to control the size of the maximum clique in order to arrive at a desired number of recipes to be batch tested together. The size of maximum clique is related to the parameter edge threshold ET. The parameter ET can be adjusted until a selected size of the maximum clique is achieved. In this scenario, at step 322, the size of maximum clique of the graph, denoted by parameter K is determined. At step 324, the parameter ET is adjusted until the size of the maximum clique of the graph is a selected value of K. At step 326, the method ends.

Example Implementation Mechanism—Hardware Overview

Some embodiments are implemented by a computer system or a network of computer systems. A computer system may include a processor, a memory, and a non-transitory computer-readable medium. The memory and non-transitory medium may store instructions for performing methods, steps and techniques described herein.

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be server computers, cloud computing computers, desktop computer systems, portable computer systems, handheld devices, networking devices or any other device that incorporates hard-wired and/or program logic to implement the techniques.

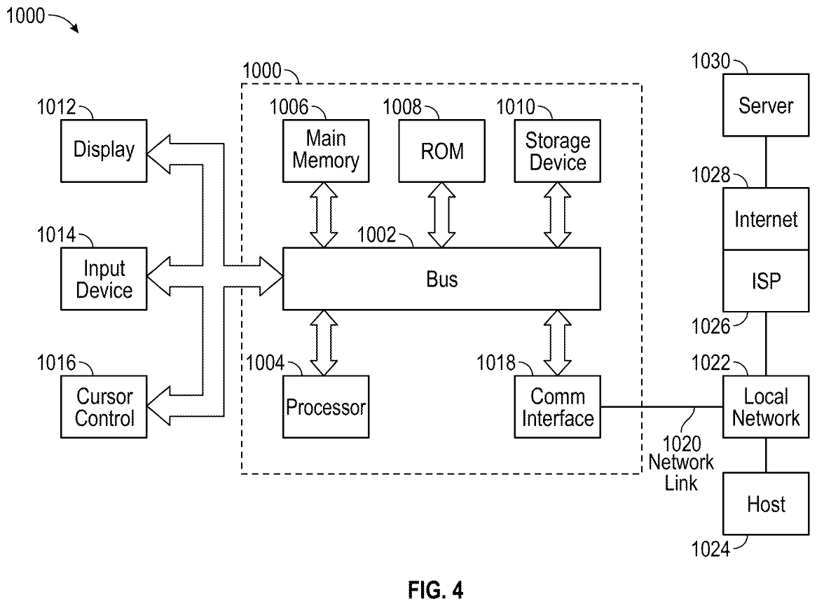

For example, FIG. 4 is a block diagram that illustrates a computer system 1000 upon which an embodiment of can be implemented. Computer system 1000 includes a bus 1002 or other communication mechanisms for communicating information, and a hardware processor 1004 coupled with bus 1002 for processing information. Hardware processor 1004 may be, for example, special-purpose microprocessor optimized for handling audio and video streams generated, transmitted or received in video conferencing architectures.

Computer system 1000 also includes a main memory 1006, such as a random access memory (RAM) or other dynamic storage devices, coupled to bus 1002 for storing information and instructions to be executed by processor 1004. Main memory 1006 also may be used for storing temporary variables or other intermediate information during execution of instructions to be executed by processor 1004. Such instructions, when stored in non-transitory storage media accessible to processor 1004, render computer system 1000 into a special-purpose machine that is customized to perform the operations specified in the instructions.

Computer system 1000 further includes a read only memory (ROM) 1008 or other static storage device coupled to bus 1002 for storing static information and instructions for processor 1004. A storage device 1010, such as a magnetic disk, optical disk, or solid-state disk is provided and coupled to bus 1002 for storing information and instructions.

Computer system 1000 may be coupled via bus 1002 to a display 1012, such as a cathode ray tube (CRT), liquid crystal display (LCD), organic light-emitting diode (OLED), or a touchscreen for displaying information to a computer user. An input device 1014, including alpha-numeric and other keys (e.g., in a touch screen display) is coupled to bus 1002 for communicating information and command selections to processor 1004. Another type of user input device is cursor control 1016, such as a mouse, a trackball, or cursor direction keys for communicating direction information and command selections to processor 1004 and for controlling cursor movement on display 1012. This input device typically has two degrees of freedom in two axes, a first axis (e.g., x) and a second axis (e.g., y), that allows the device to specify positions in a plane. In some embodiments, the user input device 1014 and/or the cursor control 1016 can be implemented in the display 1012 for example, via a touch-screen interface that serves as both output display and input device.

Computer system 1000 may implement the techniques described herein using customized hard-wired logic, one or more ASICs or FPGAs, firmware and/or program logic which in combination with the computer system causes or programs computer system 1000 to be a special-purpose machine. According to one embodiment, the techniques herein are performed by computer system 1000 in response to processor 1004 executing one or more sequences of one or more instructions contained in main memory 1006. Such instructions may be read into main memory 1006 from another storage medium, such as storage device 1010. Execution of the sequences of instructions contained in main memory 1006 causes processor 1004 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical, magnetic, and/or solid-state disks, such as storage device 1010. Volatile media includes dynamic memory, such as main memory 1006. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 1002. Transmission media can also take the form of acoustic or light waves, such as those generated during radio-wave and infra-red data communications.

Various forms of media may be involved in carrying one or more sequences of one or more instructions to processor 1004 for execution. For example, the instructions may initially be carried on a magnetic disk or solid-state drive of a remote computer. The remote computer can load the instructions into its dynamic memory and send the instructions over a telephone line using a modem. A modem local to computer system 1000 can receive the data on the telephone line and use an infra-red transmitter to convert the data to an infra-red signal. An infra-red detector can receive the data carried in the infra-red signal and appropriate circuitry can place the data on bus 1002. Bus 1002 carries the data to main memory 1006, from which processor 1004 retrieves and executes the instructions. The instructions received by main memory 1006 may optionally be stored on storage device 1010 either before or after execution by processor 1004.

Computer system 1000 also includes a communication interface 1018 coupled to bus 1002. Communication interface 1018 provides a two-way data communication coupling to a network link 1020 that is connected to a local network 1022. For example, communication interface 1018 may be an integrated services digital network (ISDN) card, cable modem, satellite modem, or a modem to provide a data communication connection to a corresponding type of telephone line. As another example, communication interface 1018 may be a local area network (LAN) card to provide a data communication connection to a compatible LAN. Wireless links may also be implemented. In any such implementation, communication interface 1018 sends and receives electrical, electromagnetic or optical signals that carry digital data streams representing various types of information.

Network link 1020 typically provides data communication through one or more networks to other data devices. For example, network link 1020 may provide a connection through local network 1022 to a host computer 1024 or to data equipment operated by an Internet Service Provider (ISP) 1026. ISP 1026 in turn provides data communication services through the world-wide packet data communication network now commonly referred to as the “Internet” 1028. Local network 1022 and Internet 1028 both use electrical, electromagnetic or optical signals that carry digital data streams. The signals through the various networks and the signals on network link 1020 and through communication interface 1018, which carry the digital data to and from computer system 1000, are example forms of transmission media.

Computer system 1000 can send messages and receive data, including program code, through the network(s), network link 1020 and communication interface 1018. In the Internet example, a server 1030 might transmit a requested code for an application program through Internet 1028, ISP 1026, local network 1022 and communication interface 1018.

The received code may be executed by processor 1004 as it is received, and/or stored in storage device 1010, or other non-volatile storage for later execution.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed is:

1. A method of selecting batches of recipes that minimizes the number of recipe components in each batch for high-throughput laboratory analysis, the method comprising:
receiving identities of a plurality of chemicals S;
randomly generating a plurality of recipes;
receiving an edge threshold parameter ET, wherein ET comprises a selected number of shared chemicals between each recipe;
randomly generating combinations C of chemicals from the pool of chemicals S, comprising C(S, ET);
generating a plurality of buckets of recipes using the combinations C, wherein each bucket comprises recipes sharing at least ET number of chemicals in common;
generating a graph having a plurality of nodes, wherein each node of the graph comprises one of the randomly generated recipes;
connecting the nodes of the graph, wherein the connected nodes comprise recipes in a single bucket;
determining a maximum clique of the graph; and
outputting the recipes in nodes of the maximum clique.

2. The method of claim 1, further comprising:
determining a size of the maximum clique comprising a constant integer K, indicating a number of recipes in the maximum clique; and
adjusting the edge threshold ET until the determined size of the maximum clique arrives at a preselected value of K.

3. The method of claim 1, further comprising tagging each recipe with a corresponding bucket number and wherein connecting the nodes further comprises pairwise connecting the nodes that share same bucket numbers.

4. The method of claim 1, further comprising:
receiving a number M, indicating number of chemicals in a recipe, wherein the plurality of recipes are randomly generated to have M number of chemicals in each recipe.

5. The method of claim 1, further comprising:
receiving a list of essential chemicals, ECS, wherein the plurality of recipes are randomly generated such that each recipe includes the essential chemicals ECS.

6. The method of claim 1, further comprising:
receiving a number M, indicating number of chemicals in each recipe; and
receiving a list of essential chemicals, ECS, wherein the plurality of recipes are randomly generated such that each recipe has M chemicals including the essential chemicals ECS.

7. The method of claim 1, further comprising:
applying a filter to the randomly generated recipes, wherein the filter excludes recipes using rare chemicals.

8. The method of claim 7, wherein the rare chemicals are identified at least in part based on constructing a frequency table, comprising frequency of occurrence of each chemical in the plurality of randomly generated recipes.

9. The method of claim 1, wherein determining maximum clique comprises applying a MaxCliqueDyn algorithm.

10. The method of claim 1, wherein instead of randomly generating the plurality of the recipes, the plurality of the recipes are received from an output of an AI model.

11. Non-transitory computer storage that stores executable program instructions that, when executed by one or more computing devices, configure the one or more computing devices to perform operations comprising:
receiving identities of a plurality of chemicals S;
randomly generating a plurality of recipes;
receiving an edge threshold parameter ET, wherein ET comprises a selected number of shared chemicals between each recipe;
randomly generating combinations C of chemicals from the pool of chemicals S, comprising C(S, ET);
generating a plurality of buckets of recipes using the combinations C, wherein each bucket comprises recipes sharing at least ET number of chemicals in common;
generating a graph having a plurality of nodes, wherein each node of the graph comprises one of the randomly generated recipes;
connecting the nodes of the graph, wherein the connected nodes comprise recipes in a single bucket;
determining a maximum clique of the graph; and
outputting the recipes in nodes of the maximum clique.

12. The non-transitory computer storage of claim 11, wherein the operations further comprise:
determining a size of the maximum clique comprising a constant integer K, indicating a number of recipes in the maximum clique; and
adjusting the edge threshold ET until the determined size of the maximum clique arrives at a preselected value of K.

13. The non-transitory computer storage of claim 11, wherein the operations further comprise tagging each recipe with a corresponding bucket number and wherein connecting the nodes further comprises pairwise connecting the nodes that share same bucket numbers.

14. The non-transitory computer storage of claim 11, wherein the operations further comprise:
receiving a number M, indicating number of chemicals in a recipe, wherein the plurality of recipes are randomly generated to have M number of chemicals in each recipe.

15. The non-transitory computer storage of claim 11, wherein the operations further comprise:
receiving a list of essential chemicals, ECS, wherein the plurality of recipes are randomly generated such that each recipe includes the essential chemicals ECS.

16. The non-transitory computer storage of claim 11, wherein the operations further comprise:
receiving a number M, indicating number of chemicals in each recipe; and
receiving a list of essential chemicals, ECS, wherein the plurality of recipes are randomly generated such that each recipe has M chemicals including the essential chemicals ECS.

17. The non-transitory computer storage of claim 11, wherein the operations further comprise:
applying a filter to the randomly generated recipes, wherein the filter excludes recipes using rare chemicals.

18. The non-transitory computer storage of claim 17, wherein the rare chemicals are identified at least in part based on constructing a frequency table, comprising frequency of occurrence of each chemical in the plurality of randomly generated recipes.

19. The non-transitory computer storage of claim 11, wherein determining maximum clique comprises applying a MaxCliqueDyn algorithm.

20. The non-transitory computer storage of claim 11, wherein instead of randomly generating the plurality of the recipes, the plurality of the recipes are received from an output of an AI model.
